(19) United States
(12) Patent Application Publication
(10) Pub. No.: US 2020/0129151 A1
(43) Pub. Date: Apr. 30, 2020

(54) METHODS AND APPARATUSES FOR ULTRASOUND IMAGING USING DIFFERENT IMAGE FORMATS

(71) Applicants: Abraham Neben, New Haven, CT (US); Karl Thiele, Andover, MA (US); Christophe Meyer, New York, NY (US); Robert Schneider, Killingworth, CT (US)

(72) Inventors: Abraham Neben, New Haven, CT (US); Karl Thiele, Andover, MA (US); Christophe Meyer, New York, NY (US); Robert Schneider, Killingworth, CT (US)

(73) Assignee: Butterfly Network, Inc., Guilford, CT (US)

(21) Appl. No.: 16/662,252

(22) Filed: Oct. 24, 2019

Related U.S. Application Data
(60) Provisional application No. 62/750,443, filed on Oct. 25, 2018.

(51) Int. Cl. A61B 8/00; A61B 8/08; G06T 7/00

(52) U.S. Cl. CPC A61B 8/4488; A61B 8/4444; G06T 7/0012; A61B 8/5207

(57) ABSTRACT

Aspects of the technology described herein include ultrasound data collection using different image formats. Some embodiments include causing, within a single imaging preset, an ultrasound device having a single transducer array to switch from a configuration to collect ultrasound data for producing ultrasound images having a first format to a configuration to collect ultrasound data for producing ultrasound images having a second format. Some embodiments include modulating, within a single imaging preset and as a function of imaging depth, a virtual apex location and/or an instantaneous transmit aperture size used for ultrasound data collection by an ultrasound device having a single transducer array.

### CROSS-REFERENCE TO RELATED APPLICATIONS

This application claims the benefit under 35 U.S.C. § 119(e) of U.S. Patent Application Ser. No. 62/750,443, filed. Oct. 25, 2018 under Attorney Docket No. B1348.70117US00, and entitled “METHODS AND APPARATUSES FOR ULTRASOUND IMAGING USING DIFFERENT IMAGE FORMATS”, which is hereby incorporated herein by reference in its entirety.

### FIELD

Generally, the aspects of the technology described herein relate to ultrasound imaging using different image formats.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method of operating an ultrasound device having a single ultrasound transducer array includes causing, within a single imaging preset, the ultrasound device to switch from a configuration to collect ultrasound data for producing ultrasound images having a first format to a configuration to collect ultrasound data for producing ultrasound images having a second format. In some embodiments, the first format is a linear format and the second format is a trapezoidal format. In some embodiments, the first format is a trapezoidal format and the second format is a sector format. In some embodiments, the first format is a trapezoidal format and the second format is a linear format. In some embodiments, the first format is a sector format and the second format is a trapezoidal format. In some embodiments, the first format is a linear format and the second format is a sector format. In some embodiments, the first format is a sector format and the second format is a linear format.

In some embodiments, causing the ultrasound device to switch from the configuration to collect ultrasound data for producing ultrasound images having the first format to the configuration to collect ultrasound data for producing ultrasound images having the second format is based on receiving a selection of a new imaging depth that exceeds a threshold imaging depth.

According to another aspect, a method of operating an ultrasound device having a single transducer array includes modulating, within a single imaging preset and as a function of imaging depth, one or more of a virtual apex location and an instantaneous transmit aperture size used for ultrasound data collection by the ultrasound device. In some embodiments, modulating one or more of the virtual apex location and the instantaneous transmit aperture size used for ultrasound data collection by the ultrasound device is based on receiving, at a processing device in operative communication with the ultrasound device, a selection of a new imaging depth that exceeds a threshold imaging depth.

Some aspects include at least one non-transitory computer-readable storage medium storing processor-executable instructions that, when executed by at least one processor, cause the at least one processor to perform the above aspects and embodiments. Some aspects include an apparatus having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example graph of transmit lines in a preset for a linear image format, in accordance with certain embodiments described herein;

FIG. 2 illustrates an example graph of transmit lines in a preset for a trapezoidal image format, in accordance with certain embodiments described herein;

FIG. 3 illustrates an example graph of transmit lines in a preset for a sector image format, in accordance with certain embodiments described herein;

FIG. 4 illustrates an example process for ultrasound imaging, in accordance with certain embodiments described herein;

FIG. 5 illustrates another example process for ultrasound imaging, in accordance with certain embodiments described herein;

FIG. 6 illustrates another example process for ultrasound imaging, in accordance with certain embodiments described herein;

FIG. 7 illustrates another example process for ultrasound imaging, in accordance with certain embodiments described herein;

FIG. 8 illustrates another example process for ultrasound imaging, in accordance with certain embodiments described herein;

FIG. 9 illustrates another example process for ultrasound imaging, in accordance with certain embodiments described herein;

FIG. 10 illustrates an example ultrasound image generated in accordance with certain embodiments described herein;

FIG. 11 illustrates another example ultrasound image generated in accordance with certain embodiments described herein;

FIG. 12 illustrates another example ultrasound image generated in accordance with certain embodiments described herein;

FIG. 13 illustrates another example ultrasound image generated in accordance with certain embodiments described herein;

FIG. 14 shows a schematic block diagram illustrating aspects of an example ultrasound system upon which various aspects of the technology described herein may be practiced; and

FIG. 15 shows a schematic block diagram illustrating aspects of another example ultrasound system upon which various aspects of the technology described herein may be practiced.

### DETAILED DESCRIPTION

Typical ultrasound systems include multiple ultrasound probes with different characteristics. For example, an ultrasound system may include a linear probe, a curvilinear probe, and a phased array probe. A linear probe may produce ultrasound images having a linear format. An ultrasound image may be considered to have a linear format when the width of the ultrasound image at the top (i.e., the edge of the ultrasound image closest to the ultrasound probe in the vertical direction) is within a threshold percentage of the width of the ultrasound image at the bottom (i.e., the edge of the ultrasound image farthest from the ultrasound probe). In some embodiments, the threshold percentage may be 10%. In some embodiments, the threshold percentage may be another value, such as 1%, 2%, 5%, 15%, 20%. A phased array probe may produce ultrasound images having a sector format. An ultrasound image may be considered to have a sector format when the width of the ultrasound image at the top is less than a threshold percentage of the width of the ultrasound image at the bottom. In some embodiments, the threshold percentage may be 10%. In some embodiments, the threshold percentage may be another value, such as 1%, 2%, 5%, 15%, 20%. A curvilinear probe may produce ultrasound images having a trapezoidal format. An ultrasound image may be considered to have a trapezoidal format when the ultrasound image does not have a linear format or a sector format. A clinician may select a particular ultrasound probe based on the probe's image format being optimal for visualizing a certain anatomy. In general, a linear image format may be optimal for shallow imaging depths, a trapezoidal image format may be optimal for intermediate or deep imaging depths, and a sector image format may be optimal for deep imaging depths.

The inventors have recognized that certain types of ultrasonic transducers, such as capacitive micromachined ultrasonic transducers (CMUTs), may have broad bandwidths, and therefore a single ultrasound probe having a single transducer array of such transducers may enable imaging across a broad frequency range. A single ultrasound probe that can image across a broad frequency range may in turn be able to image across a broad range of depths within the subject being imaged. For example, a single preset (namely, a set of imaging parameter values optimized for imaging a particular anatomy) may have a broader range of possible imaging depths compared with a preset optimized for imaging the particular anatomy on an ultrasound probe based on piezoelectric transducers. (It should be understood that as referred to herein, the imaging parameter values in a preset need not necessarily be predetermined, but may be user-defined.)

The inventors have recognized that, for a single ultrasound probe capable of imaging across a broad range of depths, it may be helpful to vary the ultrasound image format based on the imaging depth, since different image formats may be optimal for different imaging depths. Thus, the inventors have developed technology in which the image format may switch from a first format to a second format in dependence on the imaging depth selected by a user. For example, the image format may switch from a linear format to a trapezoidal format, or vice versa, if the imaging depth selected by a user crosses a threshold depth, and the image format may switch from trapezoidal format to sector format, or vice versa, if the imaging depth crosses another threshold depth. These switches in image format may occur within a single preset and with a single ultrasound probe having a single transducer array. Thus, a single ultrasound probe possessing this format switching feature may be considered to possess the functionality of a linear probe, a curvilinear probe, and a phased array probe. Switching image format may include modulating image parameters such as the virtual apex location and/or the size of the instantaneous transmit aperture used by the ultrasound device during transmits.

In some embodiments, there may be other changes in image format based on the imaging depth. For example, at one imaging depth, a processing device may generate an ultrasound image having one width at the top of the ultrasound image, and at another imaging depth, the processing device may generate an ultrasound image having a different width at the top of the ultrasound image. As another example, at one imaging depth, a processing device may generate an ultrasound image having one ratio between the width at the top of the ultrasound image and the width at the bottom of the ultrasound image, and at another imaging depth, the processing device may generate an ultrasound image having a different ratio between the width at the top of the ultrasound image and the width at the bottom of the ultrasound image. These changes in image format may occur at multiple imaging depths. To change the image format in this manner described above, the processing device may modulate the virtual apex location and/or the instantaneous transmit aperture size as a function of imaging depth.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

It may be helpful to generate ultrasound images having a linear format for shallow imaging depths. A shallow imaging depth may be used for imaging anatomical structures of interest at shallow depths. Sometimes, anatomical features of interest positioned at shallow depths may be positioned at any lateral location below the width of the transducer array. An ultrasound image having a linear format may depict regions of the subject that are below outer regions of the transducer array even at shallow depths, and thus the use of a linear image format may be beneficial for shallow imaging.

It may be helpful to generate ultrasound images having a sector format for deep imaging depths. For deep imaging depths, it may be helpful to maximize the power transmitted by the transducer array in each transmit direction. Maximizing the power generated by the transducer array may be accomplished by using substantially all of the transducer array to transmit an ultrasound beam for a given transmit direction. When the ultrasound device uses substantially all of the transducer array to transmit ultrasound beams, it may be possible to image spatial regions below and beyond outer regions of the transducer array at deep depths by steering the ultrasound beams using beamforming techniques. Steering ultrasound beams using beamforming techniques when substantially all of the transducer array is used to transmit an ultrasound beam for a given transmit direction may result in an ultrasound image having a sector image format.

It may be helpful to generate ultrasound images having a trapezoidal format for intermediate imaging depths. An ultrasound image having a trapezoidal format may depict regions of the subject that are below outer regions of the transducer array even at shallow depths, but not as shallow as with a linear format, and may also depict regions below and beyond outer regions of the transducer array at deep depths, but not as deep as with a sector format.

Switching from generating an ultrasound image having a linear format to an ultrasound image having a trapezoidal format, from trapezoidal to linear, from trapezoidal to sector, from sector to trapezoidal, from linear to sector, or from sector to linear, may include modulating the virtual apex and/or the transmit aperture used by the ultrasound device during ultrasound transmit events, referred to herein as “transmits.” An ultrasound device may use a portion of its transducer array to generate an ultrasound beam for transmission in a given direction. The portion of the ultrasound transducer array used to generate the transmitted ultrasound pulses at any instantaneous time may be referred to as the instantaneous transmit aperture. The ultrasound device may transmit multiple ultrasound beams in multiple spatial directions in order to collect ultrasound data for forming a full ultrasound image. For each transmitted ultrasound beam using a particular instantaneous transmit aperture, one can consider a line extending from the center of the instantaneous transmit aperture along the direction of the transmitted ultrasound beam. The point in space where all such lines intersect for a given group of transmitted ultrasound beams used to form an ultrasound image may be referred to as the virtual apex.

As will be described further below, generating an ultrasound image having a linear format that depicts regions of the subject that are below outer regions of the transducer array even at shallow depths may include using a virtual apex location above the skin line—meaning in a direction away from the subject—and an instantaneous transmit aperture size that is smaller than the whole transducer array and is translated across the transducer array for transmits in different directions. Generating an ultrasound image having a sector format may include using a virtual apex location that is at the skin line and an instantaneous transmit aperture size that include substantially all of the transducer array. Generating an ultrasound image having a trapezoidal format may include using a virtual apex location and instantaneous transmit aperture size that is intermediate between those used for linear and sector formats. Thus, generating ultrasound images having depth-dependent image formats may include collecting ultrasound image using depth-dependent virtual apex locations and/or instantaneous transmit aperture sizes.

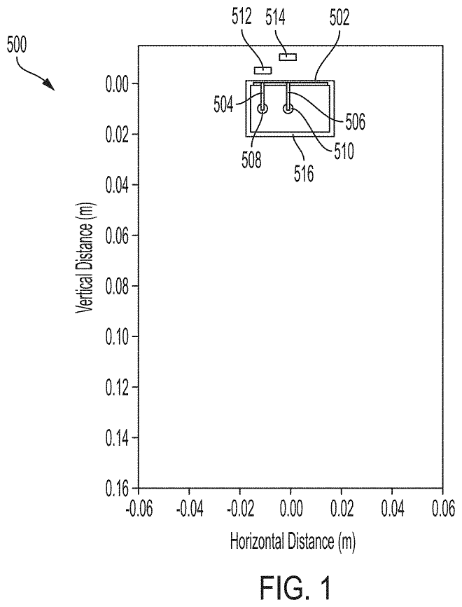

FIG. 1 illustrates an example graph 500 of transmit lines generated when using a preset for a linear image format, in accordance with certain embodiments described herein. FIG. 1 includes the locations of a transducer array 502 (shown in cross-section), a first ultrasound transmit line 504, a second ultrasound transmit line 506, and a field of view 516. The first ultrasound transmit line 504 is the leftmost transmit line generated with this preset, and has a focal location 508 and an instantaneous transmit aperture 512. The second ultrasound transmit line 506 is the centermost transmit line generated with this preset, and has a focal location 510 and an instantaneous transmit aperture 514. More transmit lines than are show in the figure are generated with this preset. The virtual apex (not shown in figure), which can be envisioned as the intersection of lines extending from the center of the instantaneous transmit apertures 512 and 514 along the directions of the first ultrasound transmit line 504 and the second ultrasound transmit line 506, respectively, is above the skin line (where the skin line is at approximately 0 m on the vertical axis). It can be further seen that the instantaneous transmit apertures 512 and 514 are subsets of the transducer array 502 that are translated across the transducer array 502. It can be further appreciated that all the transmit lines generated with this preset may enable collection of data for ultrasound images that depict regions of the subject below outer regions of the transducer array 502 even at shallow depths. The field of view 516 may be a rectangular region within which an ultrasound image is generated using data from all the transmit lines. Some received ultrasound data may correspond to data outside the field of view 516, in which case this data is not used in image reconstructed. The entire field of view 516 may not have corresponding ultrasound data.

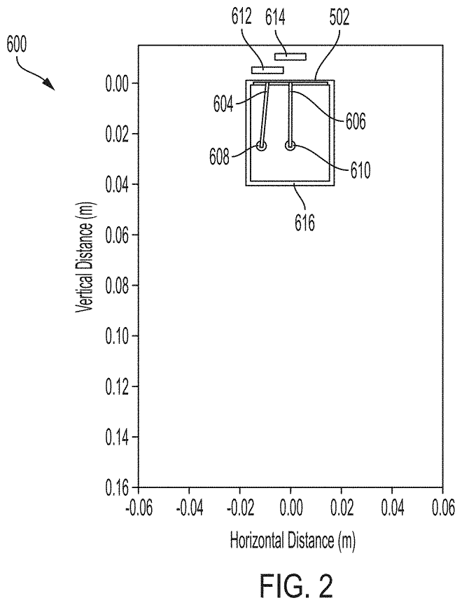

FIG. 2 illustrates an example graph 600 of transmit lines generated when using a preset for a trapezoidal image format, in accordance with certain embodiments described herein. FIG. 2 includes the locations of the transducer array 502, a first ultrasound transmit line 604, a second ultrasound transmit line 606, and a field of view 616. The first ultrasound transmit line 604 is the leftmost transmit line generated with this preset, and has a focal location 608 and an instantaneous transmit aperture 612. The second ultrasound transmit line 606 is the centermost transmit line generated with this preset, and has a focal location 610 and an instantaneous transmit aperture 614. More transmit lines than are shown in the figure are generated with this preset. The virtual apex (not shown in figure), which can be envisioned as the intersection of lines extending from the center of the instantaneous transmit apertures 612 and 614 along the directions of the first ultrasound transmit line 604 and the second ultrasound transmit line 606, respectively, is above the skin line (where the skin line is at approximately 0 m on the vertical axis) but closer to the skin line than the virtual apex of the linear format. It can be further seen that the instantaneous transmit apertures 612 and 614 are subsets of the transducer array 602 that are translated across the transducer array 602, but larger in size than the instantaneous transmit apertures 512 and 514 of the linear preset. It can be further appreciated that all the transmit lines generated with this preset may enable collection of data for ultrasound images that may depict deeper regions of the subject than ultrasound images generated with the linear preset; however, regions below outer regions of the transducer array 502 at shallow depths that may be visible with the linear preset may not be visible with the trapezoidal preset. As described above, the field of view 616 may be a rectangular region within which an ultrasound image is generated using data from all the transmit lines. Some received ultrasound data may correspond to data outside the field of view 616, in which case this data is not used in image reconstructed. The entire field of view 616 may not have corresponding ultrasound data. In trapezoidal presets, the field of view 616 may be a compromise between the desire to show the features as large as possible on the ultrasound screen, and the desire to display as much of the ultrasound data as possible out to the lower left/right fringes of the ultrasound image.

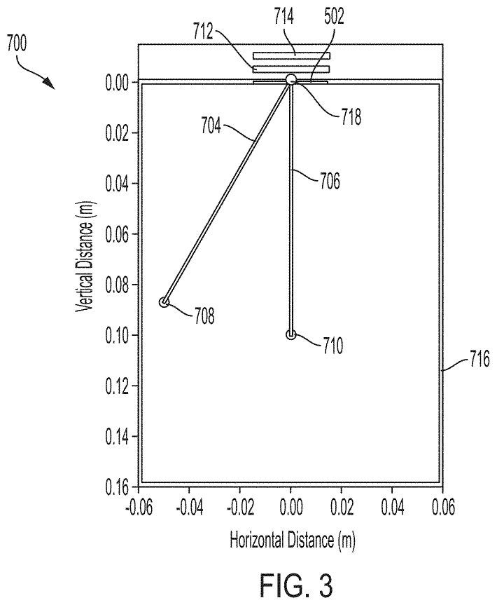

FIG. 3 illustrates an example graph 700 of transmit lines generated when using a preset for a sector image format, in accordance with certain embodiments described herein. FIG. 3 includes the locations of the transducer array 502, a first ultrasound transmit line 704, a second ultrasound transmit line 706, and a field of view 716. The first ultrasound transmit line 704 is the leftmost transmit line generated with this preset, and has a focal location 708 and an instantaneous transmit aperture 712. The second ultrasound transmit line 706 is the centermost transmit line generated with this preset, and has a focal location 710 and an instantaneous transmit aperture 714. More transmit lines than are show in the figure are generated with this preset. The virtual apex 718 is at the skin line (namely, 0 m on the vertical axis). It can be further seen that the instantaneous transmit apertures 712 and 714 encompass the entire transducer array 702. It can be further appreciated that all the transmit lines generated with this preset may enable collection of data for ultrasound images that depict deeper regions of the subject than ultrasound images generated with the trapezoidal preset; however, regions below outer regions of the transducer array 502 at shallow depths that may be visible with one or both of the linear and trapezoidal presets may not be visible with the sector preset. As described above, the field of view 616 may be a rectangular region within which an ultrasound image is generated using data from all the transmit lines. Some received ultrasound data may correspond to data outside the field of view 616, in which case this data is not used in image reconstructed. The entire field of view 616 may not have corresponding ultrasound data. Like in trapezoidal presets, in sector presets, the field of view 616 may be a compromise between the desire to show the features as large as possible on the ultrasound screen, and the desire to display as much of the ultrasound data as possible out to the lower left/right fringes of the ultrasound image.

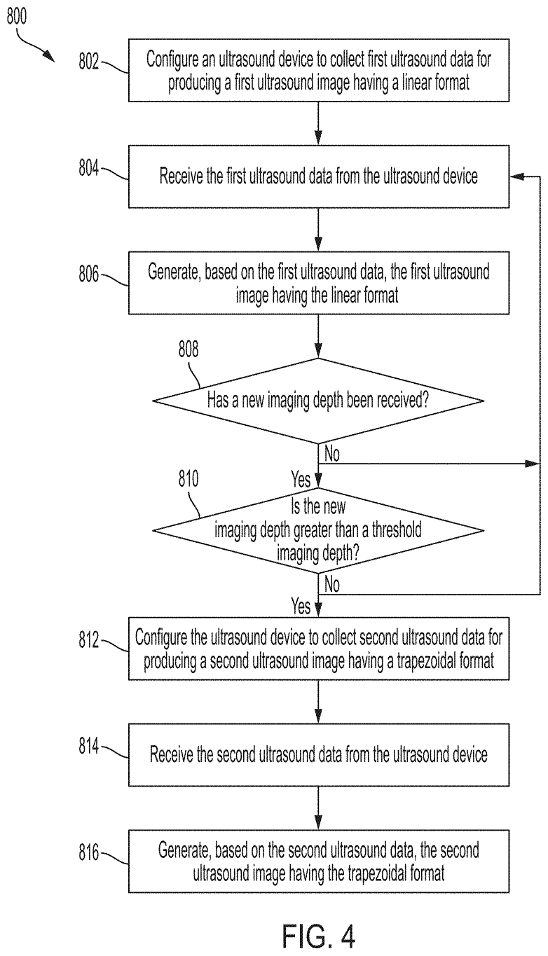

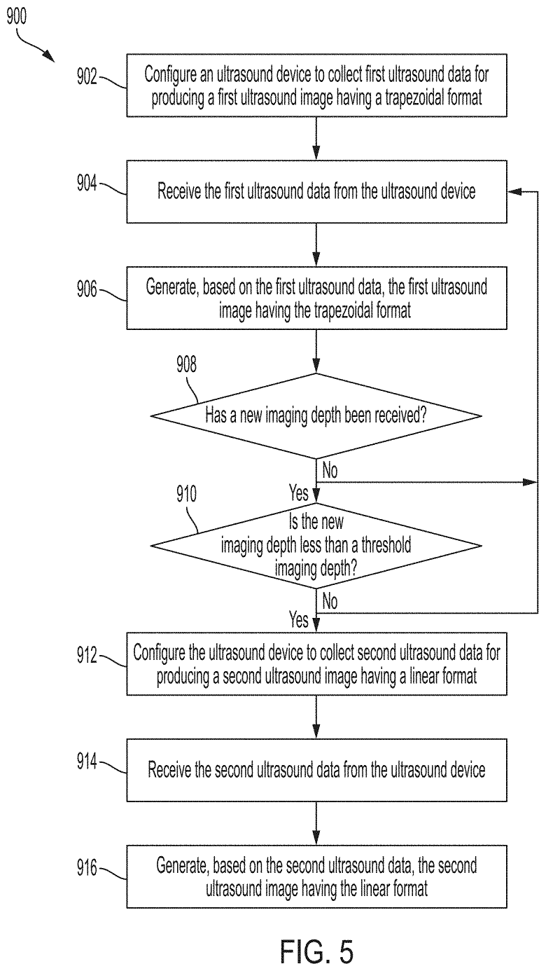

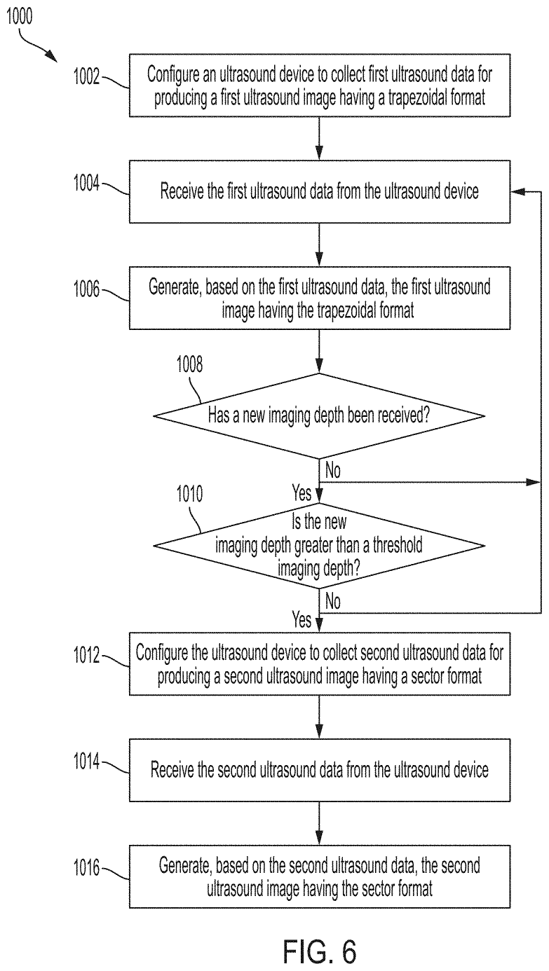

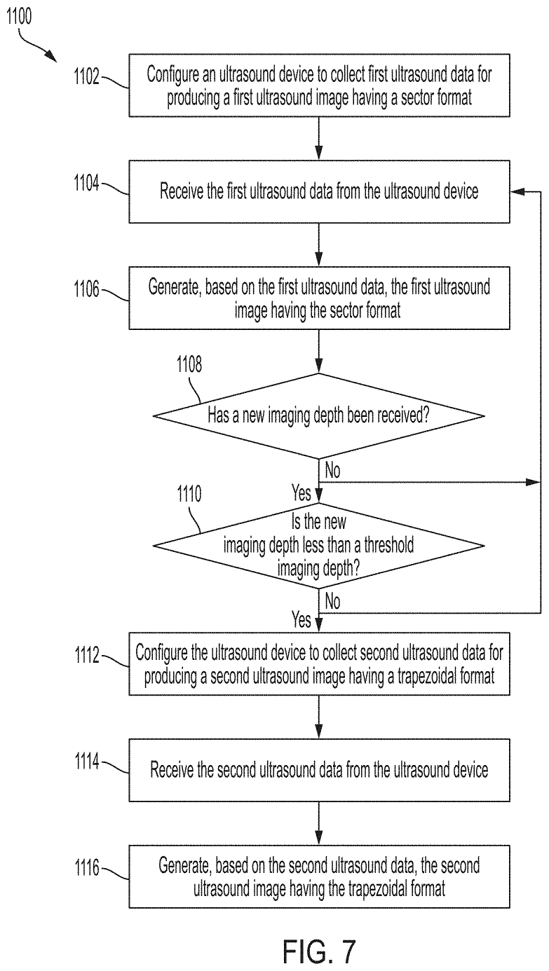

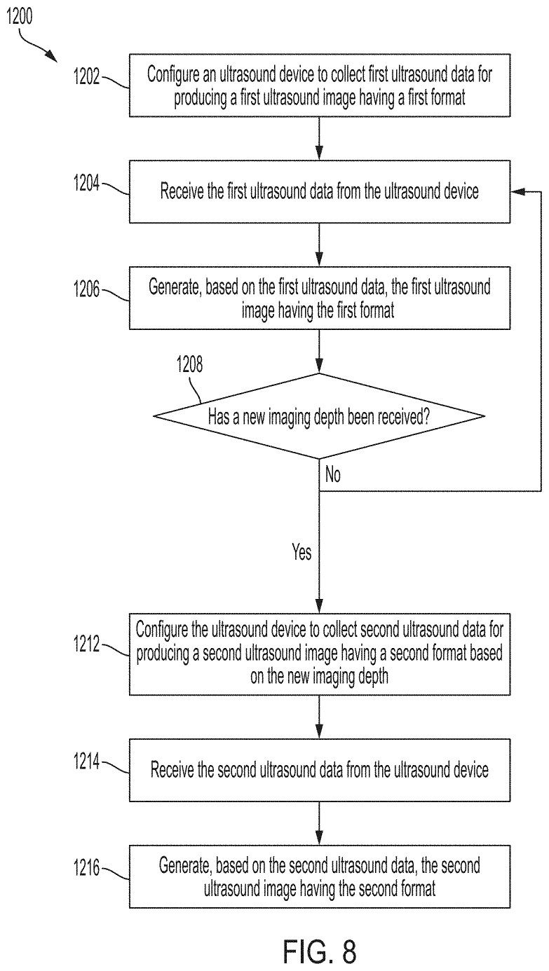

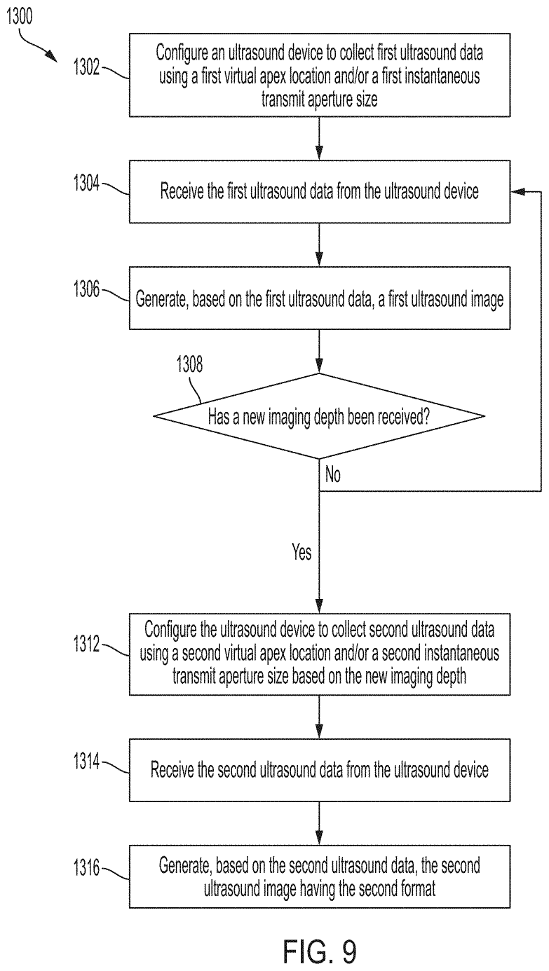

FIGS. 4-9 illustrate example processes 800, 900, 1000, 1100, 1200, and 1300 for ultrasound imaging, in accordance with certain embodiments described herein. The processes 800, 900, 1000, 1100, 1200, and 1300 are performed by a processing device in an ultrasound system. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

In act 802 of the process 800, the processing device configures the ultrasound device to collect first ultrasound data for producing a first ultrasound image having a linear format. To configure the ultrasound device to collect the first ultrasound data for producing a first ultrasound image having a linear format, the processing device may transmit commands to the ultrasound device to configure the ultrasound device with certain imaging parameters, such as virtual apex location and instantaneous transmit aperture size. The process 800 proceeds from act 802 to act 804.

In act 804, the processing device receives first ultrasound data from the ultrasound device. For example, the processing device may receive from the ultrasound device raw acoustical data, scan lines generated from raw acoustical data, and/or one or more ultrasound images generated from raw acoustical data or scan lines. The process 800 proceeds from act 804 to act 806.

In act 806, the processing device generates, based on the first ultrasound data received in act 804, a first ultrasound image having the linear format. In some embodiments, the processing device may receive raw acoustical data from the ultrasound device and generate the ultrasound image based on the raw acoustical data. In some embodiments, the processing device may receive scan lines from the ultrasound device and generate the ultrasound image based on the scan lines. In some embodiments, rather than the processing device generating the ultrasound image, the ultrasound device may generate the ultrasound image based on the first ultrasound data and transmit the ultrasound image to the processing device. The processing device may display the ultrasound image. The process 800 proceeds from act 806 to act 808.

The user may make a selection of a change in imaging depth using the processing device. For example, the user may select an imaging depth by swiping on a touch-enabled display of the processing device along a particular direction. In act 808, the processing device determines if a selection of a new imaging depth has been received. For example, the processing device may determine if a swipe along a particular direction on the touch-enabled display has been received. If a selection of a new imaging depth has not been received, the process 800 returns to act 804, where the processing device receives ultrasound data for producing an ultrasound image having a linear format. On the other hand, if a selection of a new imaging depth has been received, the process 800 proceeds to act 810. The processing device may perform the determination in act 808 periodically, and other operations of the processing device (e.g., acts 804 and 806) may occur in between such determinations.

In act 810, the processing device compares the new imaging depth to a threshold imaging depth. If the new imaging depth is greater than the threshold imaging depth, the process 800 proceeds to act 812. If the selected imaging depth is not greater than the threshold imaging depth, the process 800 proceeds back to act 804, where the processing device receives ultrasound data for producing an ultrasound image having a linear format. In some embodiments, rather than determining at act 810 if the new imaging depth is strictly greater than the threshold imaging depth, the processing device may determine at act 810 if the new imaging depth is greater than or equal to the threshold imaging depth.

Act 812 occurs if the ultrasound device was previously using an imaging depth less than or equal to the threshold imaging depth and a new imaging depth greater than the threshold imaging depth has been received. In act 812, the processing device configures the ultrasound device to collect second ultrasound data for producing a second ultrasound image having a trapezoidal format. To configure the ultrasound device to collect the second ultrasound data for producing a second ultrasound image having a trapezoidal format, the processing device may transmit commands to the ultrasound device to configure the ultrasound device with imaging parameters, such as virtual apex location and instantaneous transmit aperture size. The process 800 proceeds from act 812 to act 814.

In 814, the ultrasound device receives second ultrasound data from the ultrasound device. Further description of receiving ultrasound data may be found with reference to act 804. The process 800 proceeds from act 814 to act 816.

In act 816, the processing device generates, based on the second ultrasound data, a second ultrasound image having the trapezoidal format. Further description of generating an ultrasound image may be found with reference to act 806.

The process 900 is the same as the process 800, with the following exceptions. In act 902, the processing device configures the ultrasound device to collect first ultrasound data for producing a first ultrasound image having a trapezoidal format. In act 906, the processing device generates, based on the first ultrasound data received in act 904, the first ultrasound image having the trapezoidal format. In act 910, the processing device determines if the new imaging depth is less than a threshold imaging depth. In some embodiments, rather than determining at act 910 if the new imaging depth is strictly less than the threshold imaging depth, the processing device may determine at act 910 if the new imaging depth is less than or equal to the threshold imaging depth. In act 912, the processing device configures the ultrasound device to collect second ultrasound data for producing a second ultrasound image having a linear format. In act 916, the processing device generates, based on the second ultrasound data received in act 914, the second ultrasound image having the linear format.

The process 1000 is the same as the process 800, with the following exceptions. In act 1002, the processing device configures the ultrasound device to collect first ultrasound data for producing a first ultrasound image having a trapezoidal format. In act 1006, the processing device generates, based on the first ultrasound data received in act 1004, the first ultrasound image having the trapezoidal format. In act 1012, the processing device configures the ultrasound device to collect second ultrasound data for producing a second ultrasound image having a sector format. In act 1016, the processing device generates, based on the second ultrasound data received in act 1014, the second ultrasound image having the sector format.

The process 1100 is the same as the process 900, with the following exceptions. In act 1102, the processing device configures the ultrasound device to collect first ultrasound data for producing a first ultrasound image having a sector format. In act 1106, the processing device generates, based on the first ultrasound data received in act 1104, the first ultrasound image having the sector format. In act 1112, the processing device configures the ultrasound device to collect second ultrasound data for producing a second ultrasound image having a trapezoidal format. In act 1116, the processing device generates, based on the second ultrasound data received in act 1114, the second ultrasound image having the trapezoidal format.

The above description has described that a processing device may switch from configuring an ultrasound device to generate ultrasound images having a linear format to configuring an ultrasound device to generate ultrasound images having a trapezoidal format, or vice versa, or configuring an ultrasound device to generate ultrasound images having a trapezoidal format to configuring an ultrasound device to generate ultrasound images having a sector format, or vice versa. However, in some embodiments, the processing device may switch from configuring an ultrasound device to generate ultrasound images having a linear format to configuring an ultrasound device to generate ultrasound images having a sector format, or vice versa.

The above description has described that a processing device may configure an ultrasound device to produce ultrasound images having different image formats, such as linear, trapezoidal, or sector, based on the imaging depth. In some embodiments, there may be other changes in image format based on the imaging depth. In some embodiments, at one imaging depth, a processing device may generate a sector ultrasound image having one width at the top of the ultrasound image, and at another imaging depth, the processing device may generate a sector ultrasound image having a different width at the top of the ultrasound image. In some embodiments, at one imaging depth, a processing device may generate a sector ultrasound image having one ratio between the width at the top of the ultrasound image and the width at the bottom of the ultrasound image, and at another imaging depth, the processing device may generate a sector ultrasound image having a different ratio between the width at the top of the ultrasound image and the width at the bottom of the ultrasound image. In some embodiments, these changes in image format may occur at multiple imaging depths. In some embodiments, these changes in image format may occur at every imaging depth. In other words, every change in imaging depth may result in a change in image format. In some embodiments, these changes in image format may occur when the imaging depth changes from one range of imaging depths to another range of imaging depths, and there may be multiple such ranges.

In some embodiments, to change the image format in the manner described above, the processing device may modulate the virtual apex location and/or the instantaneous transmit aperture size as a function of imaging depth. For example, the processing device may configure the ultrasound device to use virtual apex locations that are progressively closer to the skin line for progressively deeper imaging depths and/or to use instantaneous transmit aperture sizes that are progressively larger for progressively deeper imaging depths. The processing device may configure the ultrasound device to use virtual apex locations that are progressively farther from the skin line for progressively shallower imaging depths and/or to use instantaneous transmit aperture sizes that are progressively smaller for progressively shallow imaging depths. Thus, in some embodiments, the virtual apex location and/or the instantaneous transmit aperture size may be different for every imaging depth. In some embodiments, imaging depths within a certain range may have one virtual apex location and/or one instantaneous transmit aperture size, imaging depths within another range may have another virtual apex location and/or another instantaneous transmit aperture size, and there may be any number of such ranges of imaging depths. Virtual apex locations that are progressively farther from the skin line and/or instantaneous transmit aperture sizes that are progressively smaller may result in ultrasound images having widths at the top of the ultrasound images that are progressively smaller and/or ratios of the widths at the top to the widths at the bottom of the ultrasound images that are progressively smaller.

In act 1202 of the process 1200, the processing device configures an ultrasound device to collect first ultrasound data for producing a first ultrasound image having a first format. In some embodiments, the first format may be a linear, trapezoidal, or sector image format. In some embodiments, the first format may be a format in which the ultrasound image has a particular width at the top of the ultrasound image and/or a particular ratio of the width at the top to the width at the bottom of the ultrasound image. Further description of configuring the ultrasound device to collect ultrasound data may be found with reference to act 802. The process 1200 proceeds from act 1202 to act 1204.

In act 1204, the processing device receives first ultrasound data from the ultrasound device. Further description of receiving ultrasound data may be found with reference to act 804. The process 1200 proceeds from act 1204 to act 1206.

In act 1206, the processing device generates, based on the first ultrasound data received in act 1204, the first ultrasound image having the first format. Further description of generating ultrasound images may be found with reference to act 806. The process 1200 proceeds from act 1206 to act 1208.

In act 1208, the processing device determines if a selection of a new imaging depth has been received. If a selection of a new imaging depth has not been received, the process 1200 returns to act 1204, where the processing device receives ultrasound data for producing an ultrasound image having the first format. On the other hand, if a selection of a new imaging depth has been received, the process 1200 proceeds to act 1212. Further description of determining if a selection of a new imaging depth has been received may be found with reference to act 808.

In act 1212, the processing device configures the ultrasound device to collect second ultrasound data for producing a second ultrasound image having a second format. The second format may be different from the first format. In some embodiments, the second format may be a linear, trapezoidal, or sector image format. In some embodiments, the second format may be a format in which the ultrasound image has a different width at the top of the ultrasound image than the first format and/or a different ratio of the width at the top to the width at the bottom of the ultrasound image than the first format. Further description of configuring the ultrasound device to collect ultrasound data may be found with reference to act 802. The process 1200 proceeds from act 1212 to act 1214.

In act 1214, the ultrasound device receives second ultrasound data from the ultrasound device. Further description of receiving ultrasound data may be found with reference to act 804. The process 1200 proceeds from act 1214 to act 1216.

In act 1216, the processing device generates, based on the second ultrasound data, a second ultrasound image having the second format. Further description of generating an ultrasound image may be found with reference to act 806.

In act 1302 of the process 1300, the processing device configures an ultrasound device to collect first ultrasound data using a first virtual apex location and/or a first instantaneous transmit aperture size. To configure the ultrasound device to collect the first ultrasound data using the first virtual apex location and/or the first instantaneous transmit aperture size, the processing device may transmit commands to the ultrasound device to configure the ultrasound device with the virtual apex location and/or instantaneous transmit aperture size imaging parameters. Further description of configuring the ultrasound device to collect ultrasound data may be found with reference to act 802. The process 1300 proceeds from act 1302 to act 1304.

In act 1304, the processing device receives the first ultrasound data from the ultrasound device. Further description of receiving ultrasound data may be found with reference to act 804. The process 1300 proceeds from act 1304 to act 1306.

In act 1306, the processing device generates, based on the first ultrasound data received in act 1304, a first ultrasound image. Further description of generating ultrasound images may be found with reference to act 806. The process 1300 proceeds from act 1306 to act 1308.

In act 1308, the processing device determines if a selection of a new imaging depth has been received. If a selection of a new imaging depth has not been received, the process 1300 returns to act 1304, where the processing device receives ultrasound data using the first virtual apex location and/or the first instantaneous transmit aperture size. On the other hand, if a selection of a new imaging depth has been received, the process 1300 proceeds to act 1312. Further description of determining if a selection of a new imaging depth has been received may be found with reference to act 808.

In act 1312, the processing device configures the ultrasound device to collect second ultrasound data using a second virtual apex location and/or a second instantaneous transmit aperture size based on the new imaging depth. The second virtual apex location and/or the second instantaneous transmit aperture size may be different from the first format. In some embodiments, the processing device may configure the ultrasound device to use virtual apex locations that are progressively closer to the skin line for progressively deeper imaging depths and/or to use instantaneous transmit aperture sizes that are progressively larger for progressively deeper imaging depths. The processing device may configure the ultrasound device to use virtual apex locations that are progressively farther from the skin line for progressively shallower imaging depths and/or to use instantaneous transmit aperture sizes that are progressively smaller for progressively shallow imaging depths. Thus, in some embodiments, the virtual apex location and/or the instantaneous transmit aperture size may be different for every imaging depth. In some embodiments, imaging depths within a certain range may have one virtual apex location and/or one instantaneous transmit aperture size, imaging depths within another range may have another virtual apex location and/or another instantaneous transmit aperture size, and there may be any number of such ranges of imaging depths. Further description of configuring the ultrasound device to collect ultrasound data may be found with reference to act 802. The process 1300 proceeds from act 1312 to act 1314.

In act 1314, the ultrasound device receives second ultrasound data from the ultrasound device. Further description of receiving ultrasound data may be found with reference to act 804. The process 1300 proceeds from act 1314 to act 1316.

In act 1316, the processing device generates, based on the second ultrasound data, a second ultrasound image. Further description of generating an ultrasound image may be found with reference to act 806.

It should be appreciated that in some embodiments, the changes in image format and/or imaging parameters described above may occur within a single preset and using a single ultrasound probe having a single transducer array. In other words, the image format may change in any of the manners described above without the user choosing a new preset or switching ultrasound probes. In some embodiments, the image format may change without the user making any selections aside from selecting a new imaging depth. In some embodiments, the changes in image format may occur for certain presets but not other presets. The presets where changes in image format occur may be those where the minimum imaging depth is smaller than the width of the long axis of the transducer array and the maximum imaging depth is more than twice as large as the long axis of the transducer array.

It should be appreciated that while the above description has described the processes 800, 900, 1000, 1100, 1200, and 1300 as being performed by a processing device, in some embodiments these processes may be performed by the ultrasound device that collects the ultrasound data.

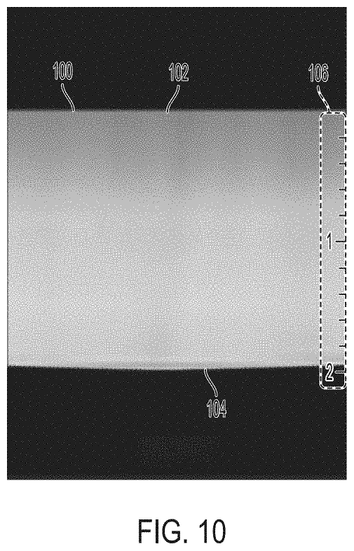

FIGS. 10-13 illustrate example ultrasound images that may be generated in accordance with certain embodiments described herein (e.g., using one or more of the processes 800, 900, 1000, 1100, 1200, and 1300). The ultrasound images are all collected by a single ultrasound transducer array on a single ultrasound device using a single imaging preset. The ultrasound images may be collected upon selection of different imaging depths by a user.

FIG. 10 illustrates an example ultrasound image 100 generated in accordance with certain embodiments described herein. The ultrasound image 100 includes a top portion 102 and a bottom portion 104. FIG. 10 further includes a ruler 106 indicating the imaging depth. In FIG. 10, the imaging depth is 2 cm, as indicated by the ruler 106. The top portion 102 of the ultrasound image 100 is approximately the same width as the bottom portion 104 of the ultrasound image 100. The ultrasound image 100 may be considered to have a linear format.

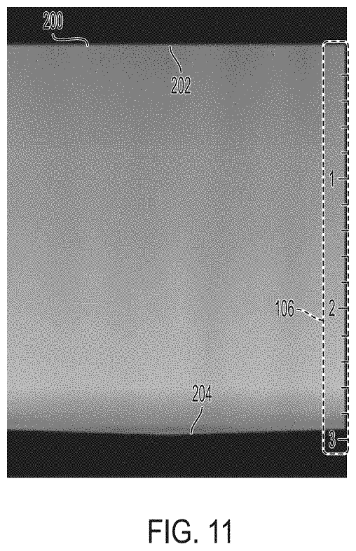

FIG. 11 illustrates another example ultrasound image 200 generated in accordance with certain embodiments described herein. The ultrasound image 200 includes a top portion 202 and a bottom portion 204. In FIG. 11, the imaging depth is 3 cm, as indicated by the ruler 106. The user may have selected the imaging depth of 3 cm after the imaging depth was 2 cm, resulting in a switch from the ultrasound image 100 to the ultrasound image 200. The top portion 202 of the ultrasound image 200 is approximately the same width as the bottom portion 204 of the ultrasound image 200. The ultrasound image 200 may also be considered to have a linear format.

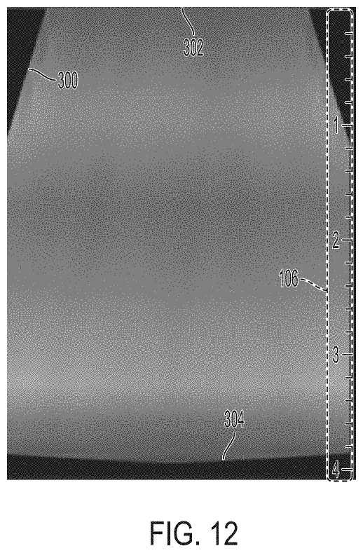

FIG. 12 illustrates another example ultrasound image 300 generated in accordance with certain embodiments described herein. The ultrasound image 300 includes a top portion 302 and a bottom portion 304. In FIG. 12, the imaging depth is 4 cm, as indicated by the ruler 106. The user may have selected the imaging depth of 4 cm after the imaging depth was 3 cm, resulting in a switch from the ultrasound image 200 to the ultrasound image 300. The top portion 302 of the ultrasound image 300 is narrower than the bottom portion 304 of the ultrasound image 300. The ultrasound image 300 may be considered to have a trapezoidal format.

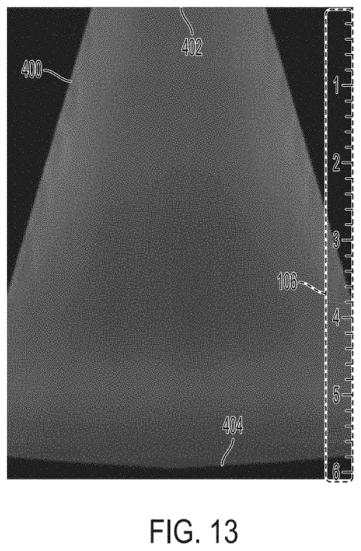

FIG. 13 illustrates another example ultrasound image 400 generated in accordance with certain embodiments described herein. The ultrasound image 400 includes a top portion 402 and a bottom portion 404. In FIG. 13, the imaging depth is 6 cm, as indicated by the ruler 106. The user may have selected the imaging depth of 6 cm after the imaging depth was 4 cm, resulting in a switch from the ultrasound image 100 to the ultrasound image 200. The top portion 402 of the ultrasound image 400 is narrower than the bottom portion 404 of the ultrasound image 400. The ultrasound image 400 may also be considered to have a trapezoidal format.

As can be appreciated from FIGS. 10-13, within the single imaging preset used to capture the ultrasound images 100, 200, 300, and 400, the image format is dependent on the imaging depth selected by the user. For imaging depths of 2 cm and 3 cm, the image format is linear, while for imaging depths of 4 cm and 6 cm, the imaging format is trapezoidal. While FIGS. 10-13 illustrate a switch in image format from linear to trapezoidal based on change in imaging depth, it should be appreciated that within a single imaging preset, a switch in image format from trapezoidal to linear, from trapezoidal to sector, from sector to trapezoidal, from linear to sector, or from sector to linear, based on imaging depth may also be implemented.

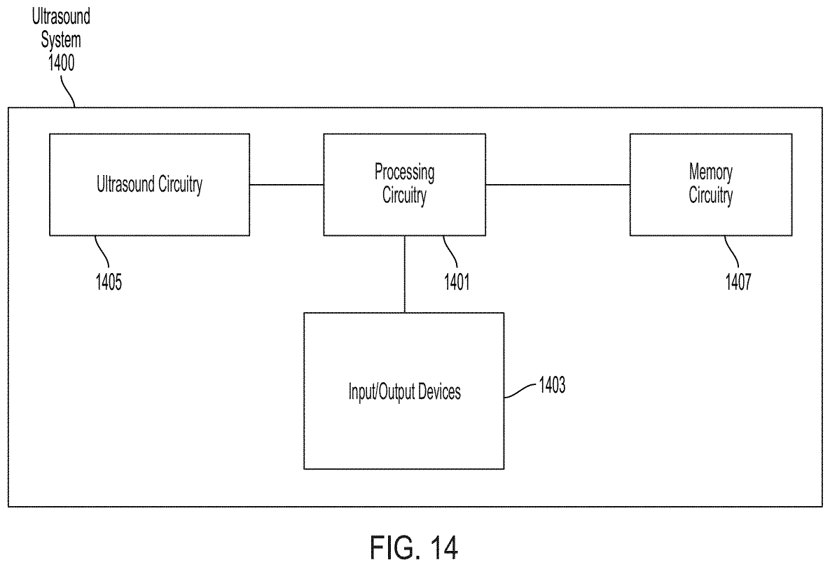

FIG. 14 shows a schematic block diagram illustrating aspects of an example ultrasound system 1400 upon which various aspects of the technology described herein may be practiced. For example, one or more components of the ultrasound system 1400 may perform any of the processes (e.g., the processes 800, 900, 1000, 1100, 1200, and 1300) described herein. As shown, the ultrasound system 1400 includes processing circuitry 1401, input/output devices 1403, ultrasound circuitry 1405, and memory circuitry 1407.

The ultrasound circuitry 1405 may be configured to generate ultrasound data that may be employed to generate an ultrasound image. The ultrasound circuitry 1405 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTS), one or more piezoelectric micromachined ultrasonic transducers (PMUTs), and/or one or more other suitable ultrasonic transducer cells. In some embodiments, the ultrasonic transducers may be formed on the same chip as other electronic components in the ultrasound circuitry 1405 (e.g., transmit circuitry, receive circuitry, control circuitry, power management circuitry, and processing circuitry) to form a monolithic ultrasound device.

The processing circuitry 1401 may be configured to perform any of the functionality described herein. The processing circuitry 1401 may include one or more processors (e.g., computer hardware processors). To perform one or more functions, the processing circuitry 1401 may execute one or more processor-executable instructions stored in the memory circuitry 1407. The memory circuitry 1407 may be used for storing programs and data during operation of the ultrasound system 1400. The memory circuitry 1407 may include one or more storage devices such as non-transitory computer-readable storage media. The processing circuitry 1401 may control writing data to and reading data from the memory circuitry 1407 in any suitable manner.

In some embodiments, the processing circuitry 1401 may include specially-programmed and/or special-purpose hardware such as an application-specific integrated circuit (ASIC). For example, the processing circuitry 1401 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

The input/output (I/O) devices 1403 may be configured to facilitate communication with other systems and/or an operator. Example I/O devices 1403 that may facilitate communication with an operator include: a keyboard, a mouse, a trackball, a microphone, a touch-enabled screen, a printing device, a display screen, a speaker, and a vibration device. Example I/O devices 1403 that may facilitate communication with other systems include wired and/or wireless communication circuitry such as BLUETOOTH, ZIGBEE, Ethernet, WiFi, and/or USB communication circuitry.

It should be appreciated that the ultrasound system 1400 may be implemented using any number of devices. For example, the components of the ultrasound system 1400 may be integrated into a single device. In another example, the ultrasound circuitry 1405 may be integrated into an ultrasound device that is communicatively coupled with a processing device that includes the processing circuitry 1401, the input/output devices 1403, and the memory circuitry 1407.

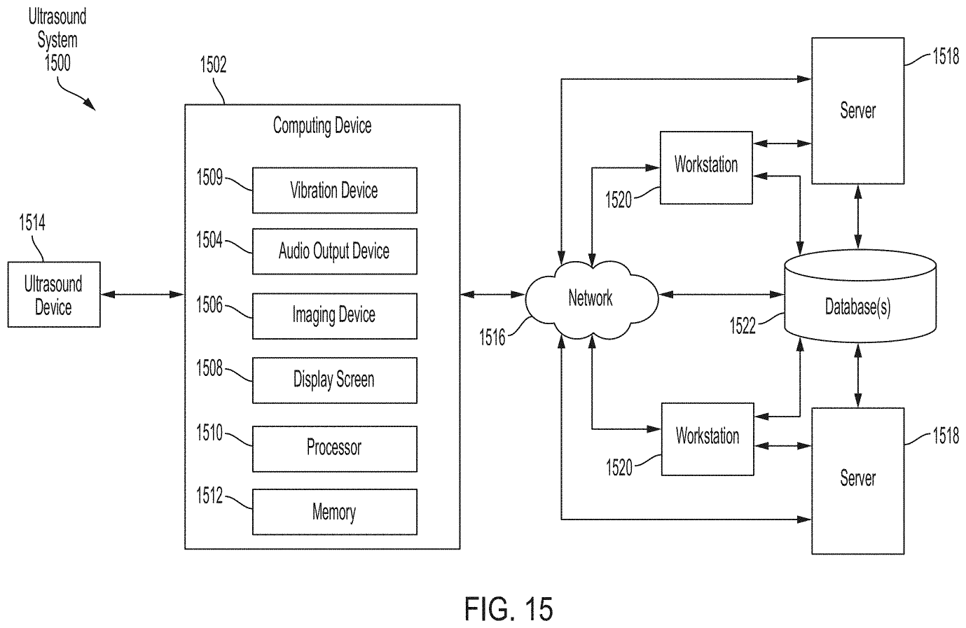

FIG. 15 shows a schematic block diagram illustrating aspects of another example ultrasound system 1500 upon which various aspects of the technology described herein may be practiced. For example, one or more components of the ultrasound system 1500 may perform any of the processes (e.g., the processes 800, 900, 1000, 1100, 1200, and 1300) described herein. As shown, the ultrasound system 1500 includes an ultrasound device 1514 in wired and/or wireless communication with a processing device 1502. The processing device 1502 includes an audio output device 1504, an imaging device 1506, a display screen 1508, a processor 1510, a memory 1512, and a vibration device 1509. The processing device 1502 may communicate with one or more external devices over a network 1516. For example, the processing device 1502 may communicate with one or more workstations 1520, servers 1518, and/or databases 1522.

The ultrasound device 1514 may be configured to generate ultrasound data that may be employed to generate an ultrasound image. The ultrasound device 1514 may be constructed in any of a variety of ways. In some embodiments, the ultrasound device 1514 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data.

The processing device 1502 may be configured to process the ultrasound data from the ultrasound device 1514 to generate ultrasound images for display on the display screen 1508. The processing may be performed by, for example, the processor 1510. The processor 1510 may also be adapted to control the acquisition of ultrasound data with the ultrasound device 1514. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

Additionally (or alternatively), the processing device 1502 may be configured to perform any of the processes (e.g., the processes 800, 900, 1000, 1100, 1200, and 1300) described herein (e.g., using the processor 1510). As shown, the processing device 1502 may include one or more elements that may be used during the performance of such processes. For example, the processing device 1502 may include one or more processors 1510 (e.g., computer hardware processors) and one or more articles of manufacture that include non-transitory computer-readable storage media such as the memory 1512. The processor 1510 may control writing data to and reading data from the memory 1512 in any suitable manner. To perform any of the functionality described herein, the processor 1510 may execute one or more processor-executable instructions stored in one or more non-transitory computer-readable storage media (e.g., the memory 1512), which may serve as non-transitory computer-readable storage media storing processor-executable instructions for execution by the processor 1510.

In some embodiments, the processing device 1502 may include one or more input and/or output devices such as the audio output device 1504, the imaging device 1506, the display screen 1508, and the vibration device 1509. The audio output device 1504 may be a device that is configured to emit audible sound such as a speaker. The imaging device 1506 may be a camera configured to detect light (e.g., visible light) to form an optical image. The display screen 1508 may be configured to display images and/or videos such as a liquid crystal display (LCD), a plasma display, and/or an organic light emitting diode (OLED) display. The display screen 1508 may be a touch-enabled screen display. The vibration device 1509 may be configured to vibrate one or more components of the processing device 1502 to provide tactile feedback. These input and/or output devices may be communicatively coupled to the processor 1510 and/or under the control of the processor 1510. The processor 1510 may control these devices in accordance with a process being executed by the process 1510 (such as the processes 800, 900, 1000, 1100, 1200, and 1300).

It should be appreciated that the processing device 1502 may be implemented in any of a variety of ways. For example, the processing device 1502 may be implemented as a handheld device such as a mobile smartphone or a tablet. Thereby, an operator of the ultrasound device 1514 may be able to operate the ultrasound device 1514 with one hand and hold the processing device 1502 with another hand. In other examples, the processing device 1502 may be implemented as a portable device that is not a handheld device such as a laptop. In yet other examples, the processing device 1502 may be implemented as a stationary device such as a desktop computer.

In some embodiments, the processing device 1502 may communicate with one or more external devices via the network 1516. The processing device 1502 may be connected to the network 1516 over a wired connection (e.g., via an Ethernet cable) and/or a wireless connection (e.g., over a WiFi network). As shown in FIG. 15, these external devices may include servers 1518, workstations 1520, and/or databases 1522. The processing device 1502 may communicate with these devices to, for example, off-load computationally intensive tasks. For example, the processing device 1502 may send an ultrasound image over the network 1516 to the server 1518 for analysis (e.g., to identify an anatomical feature in the ultrasound) and receive the results of the analysis from the server 1518. Additionally (or alternatively), the processing device 1502 may communicate with these devices to access information that is not available locally and/or update a central information repository. For example, the processing device 1502 may access the medical records of a subject being imaged with the ultrasound device 1514 from a file stored in the database 1522. In this example, the processing device 1502 may also provide one or more captured ultrasound images of the subject to the database 1522 to add to the medical record of the subject. For further description of ultrasound devices and systems, see U.S. patent application Ser. No. 15/415,434 titled “UNIVERSAL ULTRASOUND PROBE AND RELATED APPARATUS AND METHODS,” filed on Jan. 25, 2017 (and assigned to the assignee of the instant application), which is incorporated by reference herein in its entirety.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps

In some embodiments describing ranges of values, such as the ranges of imaging depths in which the shallow vs. the deep lung imaging mode are selected, a first range of values may be less than or equal to a threshold value and a second range may be greater than the threshold value. It should be understood that the range encompassing the threshold value is non-limiting, and in other embodiments the first range may be less than the value and the second range may be greater than or equal to the value. Similarly, in embodiments in which a first range of values may be less than a threshold value and a second range may be greater than or equal to the threshold value, it should be understood that in other embodiments the first range may be less than or equal to the value and the second range may be greater than the value.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A method of operating an ultrasound device having a single ultrasound transducer array, the method comprising:
causing, within a single imaging preset, the ultrasound device to switch from a configuration to collect ultrasound data for producing ultrasound images having a first format to a configuration to collect ultrasound data for producing ultrasound images having a second format.

2. The method of claim 1. wherein the first format is a linear format and the second format is a trapezoidal format.

3. The method of claim 1, wherein the first format is a trapezoidal format and the second format is a sector format.

4. The method of claim 1, wherein the first format is a trapezoidal format and the second format is a linear format.

5. The method of claim 1. wherein the first format is a sector format and the second format is a trapezoidal format.

6. The method of claim 1, wherein the first format is a linear format and the second format is a sector format.

7. The method of claim 1, wherein the first format is a sector format and the second format is a linear format.

8. The method of claim 1, wherein causing the ultrasound device to switch from the configuration to collect ultrasound data for producing ultrasound images having the first format to the configuration to collect ultrasound data for producing ultrasound images having the second format is based on receiving a selection of a new imaging depth that exceeds a threshold imaging depth.

9. A method of operating an ultrasound device having a single transducer array, the method comprising:
modulating, within a single imaging preset and as a function of imaging depth, one or more of a virtual apex location and an instantaneous transmit aperture size used for ultrasound data collection by the ultrasound device.

10. The method of claim 9, wherein modulating one or more of the virtual apex location and the instantaneous transmit aperture size used for ultrasound data collection by the ultrasound device is based on receiving, at a processing device in operative communication with the ultrasound device, a selection of a new imaging depth that exceeds a threshold imaging depth.

11. An apparatus comprising a processing device in operative communication with an ultrasound device having a single ultrasound transducer array, the processing device configured to:
cause, within a single imaging preset, the ultrasound device to switch from a configuration to collect ultrasound data for producing ultrasound images having a first format to a configuration to collect ultrasound data for producing ultrasound images having a second format.

12. The apparatus of claim 11, wherein the first format is a linear format and the second format is a trapezoidal format.

13. The apparatus of claim 11, wherein the first format is a trapezoidal format and the second format is a sector format.

14. The apparatus of claim 11, wherein the first format is a trapezoidal format and the second format is a linear format.

15. The apparatus of claim 11, wherein the first format is a sector format and the second format is a trapezoidal format.

16. The apparatus of claim 11, wherein the first format is a linear format and the second format is a sector format.

17. The apparatus of claim 11, wherein the first format is a sector format and the second format is a linear format.

18. The apparatus of claim 11, wherein the processing device is configured to cause the ultrasound device to switch from the configuration to collect ultrasound data for producing ultrasound images having the first format to the configuration to collect ultrasound data for producing ultrasound images having the second format based on receiving a selection of a new imaging depth that exceeds a threshold imaging depth.

19. An apparatus comprising a processing device in operative communication with an ultrasound device having a single ultrasound transducer array, the processing device configured to:
modulate, within a single imaging preset and as a function of imaging depth, one or more of a virtual apex location and an instantaneous transmit aperture size used for ultrasound data collection by the ultrasound device.

20. The apparatus of claim 19, wherein the processing device is configured to modulate one or more of the virtual apex location and the instantaneous transmit aperture size used for ultrasound data collection by the ultrasound device is based on receiving a selection of a new imaging depth that exceeds a threshold imaging depth.
